(19) United States
(12) Patent Application Publication
(10) Pub. No.: US 2005/0249761 A1
(43) Pub. Date: Nov. 10, 2005

(54) Topical composition comprising at least one aryl oxime and bisabolol

(76) Inventors: Joachim Buenger, Otzberg-Hering (DE); Alexandra Axt, Rossdorf (DE); Stefan Horstmann, Heppenheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/060,818

(22) Filed: Feb. 18, 2005

(30) Foreign Application Priority Data
Feb. 18, 2004 (DE) 10 2004 007 966.8

(51) Int. Cl. A61K 31/195; A61K 31/185; A61K 7/00

(52) U.S. Cl. 424/401; 514/567; 514/553

(57) ABSTRACT

The invention relates to a topical composition comprising at least one aryl oxime and bisabolol, to a mixture suitable for the preparation of the topical composition comprising at least one aryl oxime and bisabolol, and to a process for the preparation of this topical composition.

The present invention relates to a topical composition comprising at least one aryl oxime and bisabolol, to a mixture suitable for the preparation of the topical composition comprising at least one aryl oxime and bisabolol, and to a process for the preparation of this topical composition.

In many illnesses, inflammation is observed as a symptom, appearing either causally or secondarily as a consequence of pathological changes. It can likewise be caused externally by chemical or physical noxae. Inflammation is a multifunctional occurrence of various morphological and functional factors. These factors relate both to disturbances in the cellular region, in blood circulation, inflammation-induced trans- and exudation, infiltration and proliferation. As a consequence of these disturbances, further changes can occur, so that, inter alia, spongiosis, acanthosis or paraceratosis occurs.

Mediator systems are involved in the triggering, development and control of many of these processes. Thus, the lymphokinines released by sensitised T-lymphocytes are crucially involved in the cellular immune response with many biological effects (Schöpf, E., Korting, G. W. (editors) Dermatologie u. Praxis [Dermatology and Practice], Vol. 1, Thieme: Stuttgart, New York (1980)). Furthermore, the action of kinines, activated complement factors, lysosomal enzymes, cyclic nucleotides and various epidermal factors are known in this connection. A particular role is played by the prostaglandins and leukotrienes. A known example of a prostaglandin action is a chemotactic action on leukotrienes, which reduces the vascular permeability at a time subsequent to the kinines. By contrast, leukotrienes have a chemotactic action on granulocytes and influence the contractibility and permeability of vessels.

UV-B erythema is, apart from the release of histamine, mediated by the arachidonic acid cascade, with increased cyclooxygenase-mediated prostaglandin synthesis, in particular of PGE₂ and PGF₂. The lipoxygenase pathway via 5-HPETE and LTA4 results in the main elements of inflammation, such as cellular infiltration of the inflamed tissue and oedema formation (review in: Gallin, J., Goldstein, I. M., Snyderman R. (editors), Inflammation. Basic principles and clinical correlates, New York, Raven Press (1988)).

Various active ingredients are known for the treatment of inflammation. The greatest importance in the treatment of the above-mentioned mechanisms which lead to various skin diseases is possessed by corticosteroids. Weak to moderately strong corticosteroids, usually non-fluorinated derivatives of hydrocortisone, are predominantly employed for the therapy of inflammatory, allergic and pruriginous skin diseases. However, undesired side effects occur on treatment with corticosteroids, depending on the active ingredient used, the nature and duration of the treatment, and absolutely must be noted and taken into account on use of these substances (review: Symposium in Topical Corticosteroids. In: Drugs Vol. 36, 5 (1988)). For these reasons, preference is given to the use of nonsteroidal inflammation-inhibiting active ingredients, but the therapeutic efficacy of the substances known to date is very limited and usually below that of hydrocortisone. This relates to active ingredients, such as salicylic acid, acetylsalicylic acid, bufexamac, bendazac, phenylbutazone, oxyphenbutazone, diflumidone, indometacin and in some cases also antihistamines (Gloor, M.: Pharmakologie dermatologischer Externa [Pharmacology of Dermatological Topical Preparations], Springer Verlag Berlin Heidelberg New York, (1982)).

EP-A-0149 242 proposes 1-(2-hydroxyaryl)alkan-1-one oximes as active ingredients, inter alia for the treatment of skin diseases. The active ingredients can be administered orally, perlingually, rectally, parenterally, intravenously or percutaneously and as an aerosol. Pharmaceutical compositions mentioned include suspensions and emulsions, pastes, ointments, gels, creams and lotions. In one example, 2-hydroxy-5-methyllaurophenone oxime was administered intraperitoneally, and the inhibition of carrageenan oedema of the rat paw was investigated.

Besides the properties of the active ingredient and the morphological and functional state of the skin areas to be treated, it is, in particular, the properties of the vehicle used that determine the therapeutic efficacy of a topically applied medicament. Overall, the aim is an adequate, optimum concentration/time profile of the active ingredient in the afflicted skin layer.

EP-B-389 773 discloses a process for the preparation of a pharmaceutical composition having optimum bioavailability of the active ingredient 2-hydroxy-5-methyllaurophenone oxime. In this process, bile acids are used as absorbent. Bile acids which can be used are, for example, desoxy- or dehydrocholic acid or mixtures thereof in the form of salts thereof. The formulation can take place in the form of solutions, suspensions, capsules, granules, tablets or coated tablets.

DE-A41 16 123 discloses topical compositions based on 2-hydroxy-5-methyllaurophenone oxime. The areas of application mentioned are the pharmaceutical industry, human and veterinary medicine and cosmetics. Besides the active ingredient, the formulations furthermore comprise hydrating substances which normally occur in the skin and/or moisturisers and/or penetration promoters and other substances. The active-ingredient concentration is usually from 0.001 to 5%, and the above-mentioned additional substances are present in an amount of from 0.1 to 40%. The preferred hydrating substance and/or moisturiser employed is urea. Preferred penetration promoters include propylene glycol and bile acid. The topical formulation is in the form of W/O emulsions, hydrogels or mixed gels in the form of anhydrous and/or water-containing and lipophilic ointments, water-containing lipophilic ointments or nonionic creams, pastes, shaking mixtures, lotions and emulsions. The incorporation of urea was able to increase the penetration of the active ingredient into the various layers of human skin and the liberation of the active ingredient from the topical formulation. The addition of propylene glycol and sodium desoxycholate as penetration promoters was able to improve this effect further.

A major problem in the case of topical application of the above-mentioned compounds is their low solubility in cosmetic and dermatological formulations and their tendency to crystallise out. In particular, the active ingredient is soluble in emulsion systems with supply of heat, but crystallisation of this active ingredient is observed during storage after cooling both in O/W and in W/O emulsions. Even if the active ingredient remains dissolved within the emulsion systems, crystallisation in the skin layers may nevertheless occur after application of the formulation.

Numerous investigations have been carried out to obtain optimum active-ingredient compositions in which an improvement in solubility and prevention of crystallisation occur. Although aryl oximes, in particular 2-hydroxy-5-methyllaurophenone oxime, are readily soluble in various solvents, such as isopropanol, acetone, chloroform, 2-phenylethanol and Triton X100, these excipients are not suitable or only suitable to a limited extent for use for cosmetic and dermatological formulations. The excipients must therefore be non-toxic, non-carcinogenic and tolerated by the skin and should furthermore have a neutral odour. These excipients, for example solvents or emulsifiers, should furthermore be compatible with the principal component of the topical composition, i.e. an aqueous phase or an oil phase, and should not form a separate phase or result in precipitation of the active ingredient.

WO 01/89469 teaches topical formulations comprising aryl oximes and emulsifiers which increase the solubility and stability of the aryl oximes in the formulation. The emulsifiers employed are esters of lactic acid, dimers or trimers thereof with C₅- to C₁₆-acids, preferably together with further (co)emulsifiers. Liposomes are disclosed in which the aryl oximes are in liposomally encapsulated form, so that crystallisation of the aryl oxime is prevented. However, the preparation of liposomes is technically complex and thus results in an increase in the cost of the product. In addition, liposomes cannot be incorporated into formulations of all types, meaning that the freedom for the development of various formulations is greatly restricted. Furthermore, emulsifiers are always a burden for the skin, even if they are substantially physiologically tolerated. For this reason, also from basic considerations, topical formulations which if at all possible comprise no emulsifiers or, if the latter cannot be omitted, comprise them in the lowest possible concentration are desirable.

Surprisingly, it has now been found that aryl oximes can be incorporated in a stable manner in high concentration into topical formulations if they are combined with bisabolol.

This effect is based on the fact that aryl oxime(s) is (are) soluble in bisabolol. Bisabolol thus acts as solvent for aryl oxime(s). In combination with bisabolol, the aryl oxime(s) can be incorporated in high concentration into topical compositions, in particular into cosmetic and/or pharmaceutical formulations, without having to add additives, such as organic solvents or emulsifiers, for this purpose.

Like the aryl oxime, bisabolol advantageously has an inflammation-inhibiting action and thus reinforces or supplements its action. This therefore facilitates the development of simple and thus inexpensive to prepare compositions having an antiinflammatory action. Furthermore, the use of the combination of aryl oxime and bisabolol facilitates the omission of specific emulsifiers, alcohol, surfactants or the like, so that greater degrees of freedom arise in the development of the formulation and more easily tolerated products can be developed.

The topical composition according to the invention is suitable for the prophylaxis and/or treatment of skin diseases and/or inflammation reactions of the skin. Furthermore, the topical composition according to the invention can be used for the cosmetic care of the skin.

Owing to the presence of bisabolol, topical formulations can be provided in which aryl oxime(s) is (are) uniformly distributed and present in high concentration, so that, after application of the formulation to the skin, the aryl oxime(s) can be taken up in large amount thereby.

The invention therefore relates to a composition comprising at least one aryl oxime of the general formula (I) 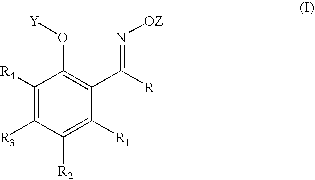
in which:
Y and Z, independently of one another, denote H, C₁₋₈-alkyl, C₂₋₁₈-alkenyl, C₂₋₁₈-carboxyalkyl, C₃₋₁₈-carboxyalkenyl or C₂₋₁₈-alkanoyl;
R denotes C₁₋₁₈-alkyl, C₂₋₁₈-alkenyl, C₃₋₈-cycloalkyl, aryl, aralkyl, heteroaryl, heteroaralkyl or fused systems;
   R₁, R₂, R₃ and R₄, independently of one another, denote H, C₁₋₂-alkyl, C₂₋₁₂-alkenyl, C₁₋₁₂-alkoxy, C₃₋₈-cycloalkoxy, aryl, aryloxy, aralkyl, heteroaryl, heteroaralkyl, carboxyl, hydroxyl, chlorine, dialkylamine or sulfonyl,
and bisabolol.

Alkyl, alkenyl, carboxyalkyl, carboxyalkenyl, alkanoyl, cycloalkyl, alkoxy, aryl, aryloxy and aralkyl may be unsubstituted or substituted. Suitable substituents of these groups are preferably alkyl, alkoxy, alkenyl, aryl, aryloxy, aralkyl, heteroaryl, heteroaralkyl, hydroxyl, carboxyl, carboxyalkyl, dialkylamine, sulfonyl and combinations thereof.

Alkyl in each case denotes straight-chain or branched alkyl and therefore preferably denotes methyl, ethyl, propyl, isopropyl, butyl, isobutyl, t-butyl, pentyl, hexyl, heptyl, octyl, nonyl, decyl, undecyl, dodecyl, tridecyl, tetra-decyl, pentadecyl, hexadecyl, heptadecyl or octadecyl.

Alkenyl means that one or more double bonds may be present in the specified alkylene.

Aryl stands for an aromatic C₆₋₂₀-hydrocarbon radical and preferably denotes phenyl.

Aralkyl denotes an aryl-substituted alkyl group and preferably has the meaning of benzyl or phenethyl.

Cycloalkyl denotes a cyclic alkyl group and is preferably cyclopropyl, cyclobutyl, cyclopentyl, cyclohexyl, cycloheptyl or cyclooctyl.

Heteroaryl stands for an aromatic ring containing hetero atoms, preferably a nitrogen-containing ring, such as pyridinyl or pyrimidinyl.

Heteroaralkyl denotes a heteroaryl-substituted alkyl group and is preferably pyridinylmethyl or pyrimidinylmethyl.

Possible fused systems are preferably the radicals naphthyl, benzofuryl, quinolinyl, indolyl or cinnolinyl.

Dialkylamine stands for NR₅R₆, where R₅ and R₆ may be identical or different and denote C₁₋₁₂-alkyl.

Z and Y are preferably, independently of one another, a hydrogen atom, a C₁₋₆-alkyl group, which can have at least one substituent selected from —OH, —COOH, —SO₃H and NR₅R₆, an alkanoyl group, represented by —C(O)R₇, in which R₇ denotes a C₁₋₆-alkyl group, which can have at least one substituent selected from —OH, —COOH and —SO₃H, or a CONHR₈ group, in which R₈ denotes a C₆-₂₀-aryl group. Z and Y are particularly preferably, independently of one another, a hydrogen atom, —(CH₂)₁₋₆COOH, —CH₂CH(OH)CH₂OH, —(CH₂)₁₋₆SO₃H, —(CH₂)₁₋₆NR₅R₆ or C(O)(CH₂)₁₋₆COOH.

The substituent R is preferably a C₁-₁₂-alkyl group, particularly preferably C₁₋₅ and C₁₁-alkyl groups.

The substituent R₁ is preferably a hydrogen or chlorine atom.

The substituent R₂ is preferably a hydrogen or chlorine atom or a C₁₋₆-alkyl group. Particular preference is given to a hydrogen atom, a chlorine atom and a methyl group.

The substituent R₃ is preferably a hydrogen atom or a C₁₋₆-alkyl group, a C₁₋₆-alkoxy group, an O-cyclohexyl group or a benzyl group.

The substituent R₄ is preferably a hydrogen or chlorine atom.

R₁, R₂, R₃ and R₄ may, if possible, preferably be substituted by —OH, —COOH, —SO₃H or —NR₅R₆ in order, for example, to increase the water solubility.

Preferred examples of aryl oximes of the formula (I) which may be present in the composition according to the invention encompass:

4-methyl-2-hydroxycaprophenone oxime, 5-methyl-2-hydroxycaprophenone oxime, 5-methyl-2-hydroxycaprophenone-(N-phenylcarbamoyl) oxime, 5-methyl-2-hydroxylaurophenone oxime (2-hydroxy-5-methyllaurophenone oxime), 3-chloro-2-hydroxycaprophenone oxime, 4-pentoxy-2-hydroxyacetophenone oxime, 4-decyloxy-2-hydroxyacetophenone oxime, 4-benzyloxy-2-hydroxyacetophenone oxime, 4-decyloxy-2-hydroxypropiophenone oxime, 4-butoxy-5-n-hexyl-2-hydroxyacetophenone oxime, 4-pentoxy-2-hydroxycaprophenone oxime, 4-decyloxy-2-hydroxycaprophenone oxime, 4-octyloxy-2-hydroxylaurophenone oxime, 4-cyclohexyloxy-2-hydroxypropiophenone oxime, 5-chloro-2-hydroxycaprophenone oxime, 3-chloro-2-hydroxylaurophenone oxime, 5-chloro-2-hydroxylaurophenone oxime, 4-butoxy-2-hydroxyacetophenone oxime, 4-dodecyloxy-2-hydroxypropiophenone oxime, 4-hexadecyloxy-2-hydroxyacetophenone oxime, 4-octadecyloxy-2-hydroxyacetophenone oxime, 4-decyloxy-2-hydroxylaurophenone oxime, and the following oxime derivatives of 2-hydroxy-5-methyllaurophenone oxime:

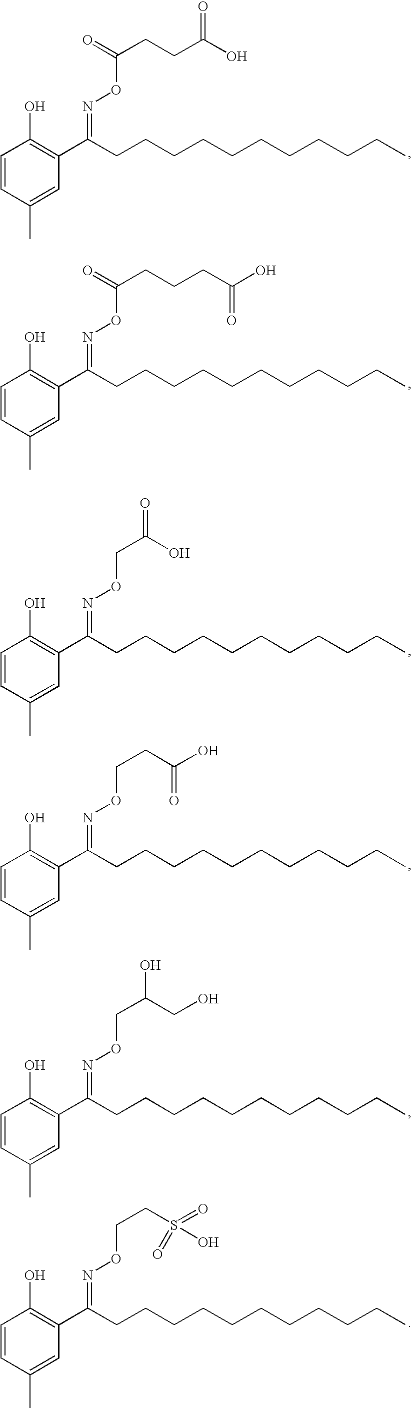

The topical composition according to the invention particularly preferably comprises 2-hydroxy-5-methyllaurophenone oxime and/or the above-mentioned oxime derivatives thereof.

The aryl oxime(s) is (are) present in the topical composition according to the invention in a sufficient amount in order to be suitable for cosmetic or dermatological use. The aryl oxime(s) is (are) usually present in the topical composition according to the invention in a total amount of from 0.001 to 5% by weight, preferably from 0.02 to 2% by weight, more preferably from 0.05 to 1.5% by weight.

Bisabolol is the principal active ingredient of camomile and is available under the trade name RonaCare® Bisabolol (Merck). The substance protects dry skin and has an antiinflammatory action. Bisabolol is usually present in the topical composition according to the invention in a total amount of from 0.05 to 30% by weight, preferably from 0.1 to 2% by weight, more preferably from 0.2 to 0.5% by weight.

Depending on the application, the topical composition according to the invention comprises adjuvants and/or excipients, such as vehicles, preservatives, stabilisers, solvents, vitamins, colorants, odour enhancers, film formers, thickeners and humectants.

Use forms of the topical composition according to the invention which may be mentioned, for example, are: solutions, suspensions, emulsions, pastes, ointments, gels, creams, lotions, surfactant-containing cleansing preparations and oils.

Ointments, pastes, creams and gels may comprise the conventional excipients, for example animal and vegetable fats, waxes, paraffins, starch, tragacanth, cellulose derivatives, polyethylene glycols, silicones, bentonites, silicic acid, talc and zinc oxide, xanthan gum, glycerol, carboxypolymethylene or mixtures of these substances.

Solutions and emulsions may comprise the conventional excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, groundnut oil, maize oil, olive oil, castor oil and sesame oil, glycerol fatty acid esters, polyethylene glycols and fatty acid esters of sorbitan, or mixtures of these substances.

The emulsions can exist in various forms. Thus, they can be, for example, an emulsion or microemulsion of the water-in-oil (W/O) type or of the oil-in-water (O/W) type, or a multiple emulsion, for example of the water-in-oil-in-water (W/O/W) type.

The composition may also be in the form of an emulsifier-free, disperse formulation. It can be, for example, a hydrodispersion or a Pickering emulsion.

The composition may also be in the form of a PIT emulsion or hydrogel and may also comprise liposomes, which include, for example, active ingredients.

Suspensions may comprise the conventional excipients, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Pastes, ointments, gels and creams may comprise the conventional excipients, for example animal and vegetable fats, waxes, paraffins, starch, tragacanth, cellulose derivatives, polyethylene glycols, silicones, bentonites, silicic acid, talc and zinc oxide, or mixtures of these substances.

Soaps may comprise the conventional excipients, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the conventional excipients, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the conventional excipients, such as synthetic oils, such as fatty acid esters, fatty alcohols, silicone oils, natural oils, such as vegetable oils and oily plant extracts, paraffin oils, lanolin oils, or mixtures of these substances.

Powders and sprays may comprise the conventional excipients, for example milk sugar, talc, silicic acid, aluminium hydroxide, calcium silicate and polyamide powder, or mixtures of these substances. Sprays may additionally comprise the conventional propellants, for example chlorofluorocarbons, propane/butane or dimethyl ether.

Further application forms of the composition according to the invention are also lipsticks, lip-care sticks, mascara, eyeliner, eye shadow, rouge, powder, emulsion and wax make-up, as well as sunscreen, pre-sun and after-sun preparations.

All compounds or components which can be used in the composition according to the invention are either known and commercially available or can be synthesised by known processes.

The formulation may comprise adjuvants which are usually used in compositions of this type, such as, for example, thickeners, softening agents, humectants, surfactants, emulsifiers, preservatives, antifoaming agents, perfumes, waxes, lanolin, propellants, dyes and/or pigments which colour the agent itself or the skin, and other ingredients usually used in cosmetics.

The dispersant or solubiliser used can be an oil, wax or other fatty body, a lower monoalcohol or a lower polyol, or mixtures thereof. The particularly preferred monoalcohols or polyols include ethanol, i-propanol, propylene glycol, glycerol and sorbitol.

Oily lotions based on natural or synthetic oils and waxes, lanolin, fatty acid esters, in particular triglycerides of fatty acids, or oily/alcoholic lotions based on a lower alcohol, such as ethanol, or a glycol, such as propylene glycol, and/or a polyol, such as glycerol, and oils, waxes and fatty acid esters, such as triglycerides of fatty acids, are a preferred embodiment.

The composition according to the invention may also be in the form of an alcoholic gel which comprises one or more lower alcohols or polyols, such as ethanol, propylene glycol or glycerol, and a thickener, such as siliceous earth. The oily/alcoholic gels also comprise natural or synthetic oil or wax.

The solid sticks consist of natural or synthetic waxes and oils, fatty alcohols, fatty acids, fatty acid esters, lanolin and other fatty bodies.

If a composition is formulated as an aerosol, the conventional propellants, such as alkanes, fluoroalkanes and chlorofluoroalkanes, are generally used.

The formulation can also be used to protect the hair against photochemical damage in order to prevent changes of colour shade, bleaching or damage of a mechanical nature. Suitable here is formulation in the form of a rinse-out shampoo, lotion, gel or emulsion, the formulation in question being applied before or after shampooing, before or after colouring or bleaching or before or after permanent waving. It is also possible to select a formulation in the form of a lotion or gel for styling and treating the hair, in the form of a lotion or gel for brushing or blow-waving, in the form of a hair lacquer, permanent waving composition, colorant or bleach for the hair. Besides aryl oxime and bisabolol, the formulation may comprise various adjuvants used in this type of composition, such as surfactants, thickeners, polymers, softening agents, preservatives, foam stabilisers, electrolytes, organic solvents, silicone derivatives, oils, waxes, antigrease agents, dyes and/or pigments which colour the composition itself or the hair, or other ingredients usually used for hair care.

The composition according to the invention can be prepared with the aid of techniques which are well known to the person skilled in the art.

For protection of the skin and/or natural or sensitised hair against sunlight, a cosmetic composition comprising aryl oxime(s) and bisabolol is applied to the skin or hair. The term sensitised hair here is taken to mean hair which has been subjected to a chemical treatment, such as a permanent-wave treatment, a colouring or bleaching process.

The topical composition according to the invention preferably furthermore comprises at least one antioxidant and/or UV filter.

Besides the known actions of the antioxidants and UV filters, such as protection against cell damage by free radicals or protection against UV radiation and the harmful action thereof, the antioxidants and/or UV filters may further stabilise the aryl oxime(s) active ingredient present. This, for example, advantageously results in an increase in the storage stability of the topical composition according to the invention.

The topical compositions according to the invention may comprise the antioxidants known from the specialist literature, for example flavonoids, coumaranones, amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), and coniferyl benzoate of benzoin resin, rutinic acid and derivatives thereof, α-glycosylrutin, ferulic acid, furfurylideneglucitol, carnosine, butylhydroxytoluene (BHT), butylhydroxyanisole, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the topical compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

In a preferred embodiment of the invention, the topical composition according to the invention comprises butylhydroxytoluene as antioxidant.

In a further preferred embodiment, the topical composition according to the invention comprises one or more compounds selected from flavonoids and/or coumaranones as antioxidant.

Flavonoids are taken to mean the glycosides of flavonones, flavones, 3-hydroxyflavones (=flavonols), aurones, isoflavones and rotenoids (Römpp Chemie Lexikon [Römpp's Lexicon of Chemistry], Volume 9, 1993). For the purposes of the present invention, however, this term is also taken to mean the aglycones, i.e. the sugar-free constituents, and the derivatives of the flavonoids and aglycones. For the purposes of the present invention, the term coumaranones is also taken to mean derivatives thereof.

Preferred flavonoids are derived from flavonones, flavones, 3-hydroxyflavones, aurones and isoflavones, in particular from flavonones, flavones, 3-hydroxyflavones and aurones.

In a further preferred embodiment, in particular if the water solubility of the flavonoids and coumaranones is to be increased, a sulfate or phosphate group is bonded to the hydroxyl groups. Suitable counterions are, for example, the ions of the alkali or alkaline earth metals, these being selected, for example, from sodium and potassium.

In a further preferred embodiment, the flavonoids are selected from the following compounds: 4,6,3′,4′-tetrahydroxyaurone, quercetin, rutin, isoquercetin, anthocyanidine (cyanidine), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin) and the sulfates and phosphates thereof.

Of the flavonoids, particular preference is given to rutin and troxerutin. Troxerutin is especially preferred.

Of the coumaranones, 4,6,3′,4′-tetrahydroxybenzyl-3-coumaranone is preferred.

The antioxidants are generally incorporated into the topical compositions according to the invention in an amount of from 0.001 to 5% by weight, preferably from 0.5 to 5% by weight.

Suitable organic UV filters are all UVA and UVB filters known to the person skilled in the art. For both UV ranges, there are many proven substances which are known from the specialist literature, for example

benzylidenecamphor derivatives, such as
3-(4′-methylbenzylidene)-dl-camphor (for example Eusolex® 6300),
3-benzylidenecamphor (for example Mexoryl® SD),
polymers of N-{(2 and 4)-[(2-oxoborn-3-ylidene)methyl]benzyl}acrylamide (for example Mexoryl® SW),
N,N,N-trimethyl-4-(2-oxoborn-3-ylidenemethyl)anilinium methylsulfate (for example Mexoryl® SK) or
α-(2-oxoborn-3-ylidene)toluene-4-sulfonic acid (for example Mexoryl® SL),

benzoyl- or dibenzoylmethanes, such as
1-(4-tert-butylphenyl)-3-(4-methoxyphenyl)propane-1,3-dione (for example Eusolex® 9020) or
4-isopropyldibenzoylmethane (for example Eusolex® 8020),

benzophenones, such as
2-hydroxy-4-methoxybenzophenone (for example Eusolex® 4360) or
2-hydroxy-4-methoxybenzophenone-5-sulfonic acid and its sodium salt (for example Uvinul® MS-40),

methoxycinnamic acid esters, such as
2-ethylhexyl p-methoxycinnamate (for example Eusolex® 2292) or
isopentyl p-methoxycinnamate, for example as a mixture of the isomers (for example Neo Heliopan® E 1000),

salicylate derivatives, such as
2-ethylhexyl salicylate (for example Eusolex® OS),
4-isopropylbenzyl salicylate (for example Megasol®) or
3,3,5-trimethylcyclohexyl salicylate (for example Eusolex® HMS),

4-aminobenzoic acid and derivatives thereof, such as
4-aminobenzoic acid,
2-ethylhexyl 4-(dimethylamino)benzoate (for example Eusolex® 6007) or
ethoxylated ethyl 4-aminobenzoate (for example Uvinul® P25),

and further substances, such as
2-ethylhexyl 2-cyano-3,3-diphenylacrylate (for example Eusolex® OCR),
2-phenylbenzimidazole-5-sulfonic acid and potassium, sodium and triethanolamine salts thereof (for example Eusolex® 232),
3,3′-(1,4-phenylenedimethylene)bis(7,7-dimethyl-2-oxobicyclo[2.2.1]hept-1-ylmethanesulfonic acid and salts thereof (for example Mexoryl® SX) or
2,4,6-trianilino-(p-carbo-2′-ethylhexyl-1′-oxy)-1,3,5-triazine (for example Uvinul® T 150).

These organic UV filters are generally incorporated into the topical compositions according to the invention in an amount of from 0.5 to 10% by weight, preferably from 1 to 8% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[(trimethylsilyl)oxy]poly[oxy(dimethyl [and about 6% of methyl[2-[p-[2,2-bis(ethoxycarbonyl]vinyl]phenoxy]-1-methyleneethyl] and about 1.5% of methyl[3-[p-[2,2-bis(ethoxycarbonyl)vinyl)phenoxy)-propenyl) and from 0.1 to 0.4% of (methylhydrogen]silylene]] (n≈60) (for example Parsol® SLX),
2,2′-methylenebis(6-(2H-benzotriazol-2-yl)-4-(1,1,3,3-tetramethylbutyl)-phenol) (for example Tinosorb® M),
2,2′-(1,4-phenylene)bis(1H-benzimidazole-4,6-disulfonic acid), monosodium salt,
2,2′-(1,4-phenylene)bis(1H-benzimidazole-5-sulfonic acid), monosodium salt,
2,2′-(1,4-phenylene)bis(1H-benzimidazole-4,6-disulfonic acid), mono-potassium salt,
2,4-bis{[4-(2-ethylhexyloxy)-2-hydroxy]phenyl}-6-(4-methoxyphenyl)-1,3,5-triazine (for example Tinosorb® S).

These organic filters are generally incorporated into the topical composition according to the invention in an amount of from 0.5 to 20% by weight, preferably from 1 to 15% by weight.

Conceivable inorganic UV filters are those from the group consisting of titanium dioxides, for example coated titanium dioxide (for example Eusolex® T-2000 or Eusolex® T-Aqua), zinc oxides (for example Sachtotec®), iron oxides and also cerium oxides. These inorganic UV filters are generally incorporated into the topical compositions according to the invention in an amount of from 0.5 to 20% by weight, preferably from 2 to 10% by weight.

Preferred UV filters are zinc oxide, titanium dioxide, 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

Particularly preferred UV filters are zinc oxide and titanium dioxide.

Of the topical compositions according to the invention comprising titanium dioxide, preference is given to those which, besides titanium dioxide, additionally comprise one or more further UV filters selected from 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

Of these compositions, particular preference is given to those which, besides titanium dioxide, additionally comprise the UV filters 2-hydroxy-4-methoxybenzophenone and/or octyl methoxycinnamate.

According to an advantageous embodiment of the invention, the topical composition according to the invention comprises, besides aryl oxime(s) and bisabolol, one or more further substance(s) having an inflammation-inhibiting action. Examples which may be mentioned here are allantoin, hespiridin, azulene, tiliroside, 5,7-dihydroxychromone and plant extracts, such as, for example, myrrh extract, rhatany root extract, sage extract, camomile extract or extracts of Emblica officinalis.

According to a further advantageous embodiment of the invention, the topical composition according to the invention comprises one or more compatible solutes. These are substances which are involved in the osmo-regulation of plants or microorganisms and can be isolated from these organisms. The generic term compatible solutes here also encompasses the osmolytes described in German Patent Application DE-A-10133202. Suitable osmolytes are, for example, the polyols, methylamine compounds and amino acids and respective precursors thereof. Osmolytes in the sense of German Patent Application DE-A-10133202 are, in particular, substances from the group consisting of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate and proline. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoine and hydroxyectoine), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula VI 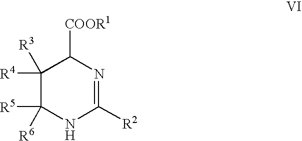
in which R¹ is a radical H or C₁₋₈-alkyl, R² is a radical H or C₁₋₄-alkyl, and R³, R⁴, R⁵ and R⁶ are each, independently of one another, a radical from the group consisting of H, OH, NH₂ and C₁₋₄-alkyl. Preference is given to the use of pyrimidinecarboxylic acids in which R²is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight. The pyrimidinecarboxylic acids are preferably employed here in ratios of from 100:1 to 1:100 with respect to the compounds of the formula I, with ratios in the range from 1:10 to 10:1 being particularly preferred.

Particular preference is given in accordance with the invention to the selection of the compatible solutes from di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP), ectoine, hydroxyectoine or mixtures thereof.

All compounds or components which can be used in the compositions are either known and commercially available or can be synthesised by known processes.

If the topical composition according to the invention comprises aryl oxime(s) and bisabolol in combination with antioxidants and/or UV filters, osmolytes and/or further inflammation inhibitor(s), a particularly strong inflammation-inhibiting and skin-calming action arises for the topical composition.

The aryl oxime(s) and bisabolol are preferably mixed with one another before incorporation. The mixing is preferably carried out with stirring and at an elevated temperature. The temperature during the mixing operation is preferably from 20 to 80° C., more preferably from 40 to 60° C. The mixture is particularly preferably prepared by dissolving the aryl oxime(s) in bisabolol.

The mixture obtained is stable on storage. The mixture can thus be employed and marketed as a precursor for the preparation of the topical composition according to the invention and then incorporated into the topical composition by the manufacturer of the topical composition according to the invention as a mixture in a simple manner by mixing, for example by simple stirring-in. The invention therefore also relates to a mixture comprising at least one aryl oxime of the formula (I) and bisabolol which is suitable for the preparation of the topical composition.

The mixture according to the invention preferably comprises from 1 to 20% by weight of aryl oxime(s) and from 20 to 99% by weight of bisabolol.

It may be particularly advantageous here for the mixture to comprise from 5 to 15% by weight, preferably from 7 to 12% by weight and particularly preferably about 10% by weight of aryl oxime(s) and from 80 to 95% by weight, preferably from 88 to 93% by weight and particularly preferably about 90% by weight of bisabolol.

In another variant of the invention, the mixture, besides the bisabolol and the aryl oxime(s), comprises further oils which are readily miscible with these constituents. Preference is given to natural oils, such as, in particular, castor oil or groundnut oil. Also suitable are oils such as Cetearyl Isononanoate, Butylphthalimide (and) Isopropylphthalimide, Isopropyl Isostearate, Ethoxydiglycol or Ethyl Butylacetylaminopropionate (names corresponding to the INCI nomenclature). The additional oils may be present in amounts of from 0 to 79% by weight, it being preferred in accordance with the invention for the additional oil to be present in the mixture to the extent of less than 50% by weight, preferably at most 30% by weight, but at least in an amount of 1% by weight, preferably 5% by weight. In this case, it is preferred for the mixture to comprise from 5 to 15% by weight, preferably from 7 to 12% by weight and particularly preferably about 10% by weight of aryl oxime(s) and from 35 to 94% by weight, preferably from 58 to 88% by weight and particularly preferably about 80% by weight of bisabolol.

In chemical engineering, the term “mixing” is taken to mean basic operations which serve for very substantial homogenisation of substances. The aim is to combine substance streams in such a way that a highly uniform composition of the individual components arises in part-volumes of the resultant mixture.

A specific form of mixing is homogenisation. This is taken to mean mixing of phases which are not miscible with one another per se. Homogenisation is accordingly taken to mean a change in the state of distribution and particle size of the inner phase of emulsions and suspensions, so that, considered in microscopic terms, a homogeneous system is formed and the distributed phase does not settle or cream without the action of external forces.

Dispersal is taken to mean mixing of a substance system consisting of two or more phases in which one substance (disperse phase) is distributed (dispersed) in very fine form in another substance (dispersion medium). Both the particles of the disperse phase and the dispersion medium may be solid, liquid or gaseous. Examples of dispersions are aerosols, emulsions, suspensions and colloids.

Another type of mixing which is usual in the preparation of cosmetics consists in emulsification. This is taken to mean mixing of two liquids which are insoluble or only sparingly soluble in one another, one of which is finely distributed in the other. The outer phase is referred to as the continuous phase or the dispersion medium, the liquid distributed therein is referred to as the inner, discontinuous or disperse phase. Cosmetic emulsions usually consist of an aqueous polar phase and a nonpolar oil phase.

Suspension on the other hand is taken to mean the distribution of very small, but non-molecular particles of a solid or liquid. Like emulsions, suspensions are usually optically cloudy and tend to settle under the influence of gravity.

The above-mentioned mixing processes are suitable for the preparation of the topical composition according to the invention. The topical composition according to the invention is particularly preferably prepared by homogenisation, dispersal or emulsification.

The invention also relates to a process for the preparation of the topical composition according to the invention which is characterised in that the aryl oxime(s) and bisabolol are incorporated in the form of a mixture.

The invention furthermore relates to the use of the topical composition according to the invention comprising at least one aryl oxime and bisabolol for the prophylaxis and/or treatment of skin diseases and/or inflammation reactions of the skin.

The invention furthermore relates to the use of the topical composition according to the invention for the cosmetic care of the skin.

The invention likewise relates to the use of the mixture comprising at least one aryl oxime of the formula (I) and bisabolol for the preparation of the topical composition according to the invention.

The topical composition according to the invention is suitable for the prophylaxis, care and/or treatment of skin diseases and/or inflammation reactions of the skin. In particular, the following skin diseases and inflammation reactions of the skin may be mentioned:
irritative dermatitis,
toxic dermatitis,
allergic diseases of the skin or skin adnexa,
inflammatory diseases of the skin or skin adnexa,
UV dermatitis and
various forms of eczema.

The working examples explain the invention without being restricted thereto.

### EXAMPLE 1

Mixtures of Aryl Oxime and Bisabolol

Mixtures of 2-hydroxy-5-methyllaurophenone oxime and bisabolol are prepared by dissolving the respective amount of 2-hydroxy-5-methyllaurophenone oxime in the respective amount of bisabolol with stirring.

Ingredients (% by wt.)

| | | | | | |
|---|---|---|---|---|---|
| RonaCare ® LPO (2- | 10.0 | 8.0 | 5.0 | 20.0 | 17.0 |
| hydroxy-5-methyllauro- | | | | | |
| phenone oxime) | | | | | |
| RonaCare ® bisabolol | 90.00 | 92.0 | 95.0 | 80.0 | 83.0 |
| RonaCare ® LPO (2- | 10.0 | 12.0 | 15.0 | 10.0 | 5.0 |
| hydroxy-5-methyllauro- | | | | | |
| phenone oxime) | | | | | |
| RonaCare ® bisabolol | 45.0 | 50.0 | 66.0 | 80.0 | 70.0 |
| Castor oil | 45.0 | 35.0 | 29.0 | 10.0 | 25.0 |

For the preparation of the topical compositions below, use is in each case made of mixtures in which 2-hydroxy-5-methyllaurophenone oxime and bisabolol are present in the respective desired weight ratio to one another.

### EXAMPLE 2

Topical Composition as O/W Emulsion

| | | Sup- | | % by | % by |
|---|---|---|---|---|---|
| | Raw material | plier | INCI name | wt. | wt. |
| A | Emulgtor E | (1) | STEARETH-10, | 2.00 | 2.00 |
| | 2155 | | STEARETH-7, | | |
| | | | STEARYL ALCOHOL | | |
| | Teginacid H | (1) | GLYCERYL | 2.00 | 2.00 |
| | | | STEARATE, CETETH- | | |
| | | | 20 | | |
| | Luvitol EHO | (2) | CETEARYL | 10.00 | 10.00 |
| | | | OCTANOATE | | |
| | Imwitor 900 | (3) | GLYCERYL | 3.00 | 3.00 |
| | | | STEARATE | | |
| | Cetiol | (4) | OLEYL OLEATE | 5.00 | 5.00 |
| | Lunacera M | (5) | MICROWAX | 1.00 | 1.00 |
| | Miglyol | (3) | CAPRYLIC/CAPRIC | 3.00 | 3.00 |
| | 812 N | | TRIGLYCERIDE | | |
| | Abil 100 | (1) | DIMETHICONE | 0.50 | 0.50 |
| | RonaCare ® | (6) | BISABOLOL | 0.30 | 0.30 |
| | bisabolol | | | | |
| | RonaCare ® | (6) | LAURYL P-CRESOL | 0.05 | 1.0 |
| | LPO | | KETOXIME | | |
| | RonaCare ® | (6) | TOCOPHERYL | 0.50 | 0.50 |
| | tocopherol | | ACETATE | | |
| | acetate | | | | |
| | Propyl 4- | (6) | PROPYLPARABEN | 0.05 | 0.05 |
| | hydroxy- | | | | |
| | benzoate | | | | |
| B | Demin. water | | AQUA | to 100 | to 100 |
| | 1,2-Pro- | (6) | PROPYLENE | 4.00 | 4.00 |
| | panediol | | GLYCOL | | |
| | Methyl 4- | (6) | METHYLPARABEN | 0.15 | 0.15 |
| | hydroxy- | | | | |
| | benzoate | | | | |

(1) Degussa-Goldschmidt AG,
(2) BASF AG,
(3) Sasol Germany GmbH,
(4) Cognis GmbH,
(5) H. B. Fuller GmbH,
(6) Merck KGaA/Rona ®

Phase A is warmed to 75° C. and phase B is warmed to 80° C. B is added to A with stirring. The mixture is subsequently homogenised (Ultra-Turrax) and cooled.

### EXAMPLE 3

Topical Composition as Sprayable After-Sun Lotion

| | | | | % by |
|---|---|---|---|---|
| | Raw material | Supplier | INCI name | wt. |
| A | Demin. water | | AQUA | to 100 |
| | Glycerol (87%) | (1) | GLYCERIN | 4.00 |
| | LaraCare A-200 | (2) | GALACTOARABINAN | 0.25 |
| B | Mirasil DM 100 | (3) | DIMETHICONE | 1.00 |
| | Cetiol OE | (4) | DICAPRYLYL | 3.00 |
| | | | ETHER | |
| | RonaCare ® | (1) | BISABOLOL | 0.50 |
| | bisabolol | | | |
| | RonaCare ® LPO | (1) | LAURYL P-CRESOL | 0.05 |
| | | | KETOXIME | |
| C | Pemulen TR-2 | (5) | ACRYLATES/C10-30 | 0.15 |
| | | | ALKYL ACRYLATE | |
| | | | CROSSPOLYMER | |
| D | Demin. water | | AQUA | 3.00 |
| | Tris(hydroxy- | (1) | TROMETHAMINE | 0.15 |
| | methyl)amino- | | | |
| | methane | | | |

(1) Merck KGaA/Rona ®,
(2) Larex,
(3) Rhodia GmbH,
(4) Cognis GmbH,
(5) Noveon

Phases A, B and D are prepared separately, phase C is dispersed in phase B. The mixture of B and C is added to A with stirring, neutralised using D and homogenised (Ultra-Turrax).

### EXAMPLE 4

Topical Composition as Hand and Nail Cream

| | | | | % by |
|---|---|---|---|---|
| | Raw material | Supplier | INCI name | wt. |
| A | Liquid paraffin | (1) | PARAFFINUM | 2.00 |
| | | | LIQUIDUM | |
| | Arlamol HD | (2) | ISOHEXADECANE | 2.00 |
| | Isopropyl | (3) | ISOPROPYL | 3.00 |
| | palmitate | | PALMITATE | |
| | Soya bean oil | (4) | GLYCINE SOJA | 0.50 |
| | | | (SOYBEAN OIL) | |
| | Mirasil DM 350 | (5) | DIMETHICONE | 1.00 |
| | Lanette O | (3) | CETEARYL ALCOHOL | 1.00 |
| | Span 60 | (2) | SORBITAN | 1.50 |
| | | | STEARATE | |
| | Montanov 68 | (6) | CETEARYL | 4.00 |
| | | | ALCOHOL, | |
| | | | CETEARYL | |
| | | | GLUCOSIDE | |
| | RonaCare ® | (1) | BISABOLOL | 0.30 |
| | bisabolol | | | |
| | RonaCare ® LPO | (1) | LAURYL P-CRESOL | 0.015 |
| | | | KETOXIME | |
| B | Demin. water | | AQUA | to 100 |
| | Glycerol (87%) | (1) | GLYCERIN | 10.00 |
| | Panthenol- D | (7) | PANTHENOL | 0.50 |
| | RonaCare ® biotin | (1) | BIOTIN | 0.05 |
| | Preservative | | | q.s. |
| C | Rhodicare S | (5) | XANTHAN GUM | 0.30 |
| D | Fragrance Bianca | (8) | PARFUM | 0.20 |

(1) Merck KGaA/Rona ®,
(2) Uniqema,
(3) Cognis GmbH,
(4) Gustav Heess GmbH,
(5) Rhodia GmbH,
(6) Seppic,
(7) Hoffmann-La Roche AG,
(8) Symrise

Phases A and B are each warmed to 75° C. Firstly C is added to B with stirring while maintaining the temperature and, when a homogeneous mixture has been obtained, A is added. The mixture is subsequently homogenised (Ultra-Turrax) and cooled.

### EXAMPLE 5

Topical Composition as W/O Emulsion

| | | | | % by |
|---|---|---|---|---|
| | Raw material | Supplier | INCI name | wt. |
| A | RonaCare ® LPO | (1) | LAURYL P-CRESOL | 0.05 |
| | | | ETOXIME | |
| | RonaCare ® | (1) | BISABOLOL | 0.50 |
| | bisabolol | | | |
| | RonaCare ® LPO | (1) | LAURYL P-CRESOL | 0.05 |
| | | | KETOXIME | |
| | Abil EM 90 | (2) | CETYL PEG/PPG-10/1 | 2.00 |
| | | | DIMETHICONE | |
| | Beeswax white | (1) | CERA ALBA | 1.00 |
| | Cutina HR | (3) | HYDROGENATED | 0.50 |
| | | | CASTOR OIL | |
| | Liquid paraffin | (1) | PARAFFINUM | 9.50 |
| | | | LIQUIDUM (MINERAL | |
| | | | OIL) | |
| | Tegosoft DC | (2) | DECYL COCOATE | 8.00 |
| | Mirasil CM 5 | (4) | CYCLOMETHICONE | 6.00 |
| B | Sodium chloride | (1) | SODIUM CHLORIDE | 0.50 |
| | Glycerol (87%) | (1) | GLYCERIN | 3.00 |
| | Titriplex III | (1) | DISODIUM EDTA | 0.10 |
| | Demin. water | | AQUA | to 100 |
| | Preservatives | | | q.s. |
| C | Emblica ® | (1) | PHYLLANTHUS | 0.50 |
| | | | EMBLICA FRUIT | |
| | | | EXTRACT | |
| | Demin. water | | AQUA | 10.00 |
| D | Sodium hydroxide | (1) | SODIUM HYDROXIDE | 0.30 |
| | soln. (10%) | | | |
| E | Ethanol 96% | (1) | ALCOHOL | 5.00 |
| | Fragrance | | PARFUM | q.s. |

(1) Merck KGaA/Rona ®,
(2) Degussa-Goldschmidt AG,
(3) Cognis GmbH,
(4) Rhodia GmbH

Phases A and B are each warmed to 80° C. B is added to A with stirring and homogenised. The pH of C is adjusted to about 5.5 using D. The mixture of phases C/D and E is subsequently added to the mixture of phases A/B, homogenised at about 30° C. (Ultra-Turrax) and stirred until cooled to room temperature.

### EXAMPLE 6

Topical Composition as Emulsifier-Free Emulsion

| | | Sup- | | % by | % by |
|---|---|---|---|---|---|
| | Raw material | plier | INCI name | wt. | wt. |
| A | RonaCare ® | (1) | BISABOLOL | 0.20 | 0.20 |
| | bisabolol | | | | |
| | RonaCare | (1) | LAURYL | 0.04 | 0.10 |
| | LPO | | P-CRESOL | | |
| | | | KETOXIME | | |
| | Tegosoft DEC | (2) | DIETHYLHEXYL | 4.50 | 4.50 |
| | | | CARBONATE | | |
| | Prisorine 2039 | (3) | ISOSTEARYL | 3.00 | 3.00 |
| | | | ISOSTEARATE | | |
| | Ceraphyl 368 | (4) | ETHYLHEXYL | 3.80 | 3.80 |
| | | | PALMITATE | | |
| B | Aristoflex AVC | (5) | AMMONIUM | 1.00 | 1.00 |
| | | | ACRYLOYL- | | |
| | | | DIMETYL | | |
| | | | TAURATE/VP | | |
| | | | COPOLYMER | | |
| C | Demin. water | | AQUA | to 100 | to 100 |
| | 1,2-Propanediol | (1) | PROPYLENE | 5.00 | 5.00 |
| | | | GLYCOL | | |
| | Preservative | | | q.s. | q.s. |
| | Urea | (1) | UREA | 5.00 | 5.00 |
| | D-Panthenol | (6) | PANTHENOL | 0.20 | 0.20 |

(1) Merck KGaA/Rona ®,
(2) Degussa-Goldschmidt AG,
(3) Uniqema Chemie GmbH,
(4) ISP Global Technologies,
(5) Clariant GmbH,
(6) Hoffmann-La Roche AG

The constituents of phases A and B are each mixed separately from one another, then phase B is added to phase A. Phase C is added to the mixture of phases A and B with stirring.

The entire disclosures of all applications, patents and publications, cited herein and of corresponding German application No. 102004007966.8, filed Feb. 18, 2004 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A topical composition comprising bisabolol and at least one aryl oxime of formula (I) 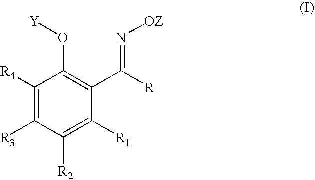
in which:
Y and Z, independently of one another, denote H, C₁₋₁₈-alkyl, C₂-₁₈-alkenyl, C₂₋₈-carboxyalkyl, C₃₋₁₈-carboxyalkenyl or C₂₋₁₈-alkanoyl;
R denotes C₁₋₁₈-alkyl, C₂₋₁₈-alkenyl, C₃₋₈-cycloalkyl, aryl, aralkyl, heteroaryl, heteroaralkyl or a fused system;
R₁, R₂, R₃ and R₄, independently of one another, denote H, C₁₋₁₂-alkyl, C₂₋₁₂-alkenyl, C₁₋₁₂-alkoxy, C₃₋₈-cycloalkoxy, aryl, aryloxy, aralkyl, heteroaryl, heteroaralkyl, carboxyl, hydroxyl, chlorine, dialkylamine or sulfonyl.

2. A topical composition according to claim 1, wherein at least one aryl oxime of formula (I) is 4-methyl-2-hydroxycaprophenone oxime, 5-methyl-2-hydroxycaprophenone oxime, 5-methyl-2-hydroxycaprophenone-(N-phenylcarbamoyl) oxime, 5-methyl-2-hydroxylaurophenone oxime (2-hydroxy-5-methyllaurophenone oxime), 3-chloro-2-hydroxycaprophenone oxime, 4-pentoxy-2-hydroxyacetophenone oxime, 4-decyloxy-2-hydroxyacetophenone oxime, 4-benzyloxy-2-hydroxyacetophenone oxime, 4-decyloxy-2-hydroxypropiophenone oxime, 4-butoxy-5-n-hexyl-2-hydroxyacetophenone oxime, 4-pentoxy-2-hydroxycaprophenone oxime, 4-decyloxy-2-hydroxycaprophenone oxime, 4-octyloxy-2-hydroxylaurophenone oxime, 4-cyclohexyloxy-2-hydroxypropiophenone oxime, 5-chloro-2-hydroxycaprophenone oxime, 3-chloro-2-hydroxylaurophenone oxime, 5-chloro-2-hydroxylaurophenone oxime, 4-butoxy-2-hydroxyacetophenone oxime, 4-dodecyloxy-2-hydroxypropiophenone oxime, 4-hexadecyloxy-2-hydroxyacetophenone oxime, 4-octadecyloxy-2-hydroxyacetophenone oxime, 4-decyloxy-2-hydroxylaurophenone oxime, or one of the following oxime derivatives of 2-hydroxy-5-methyllaurophenone oxime:

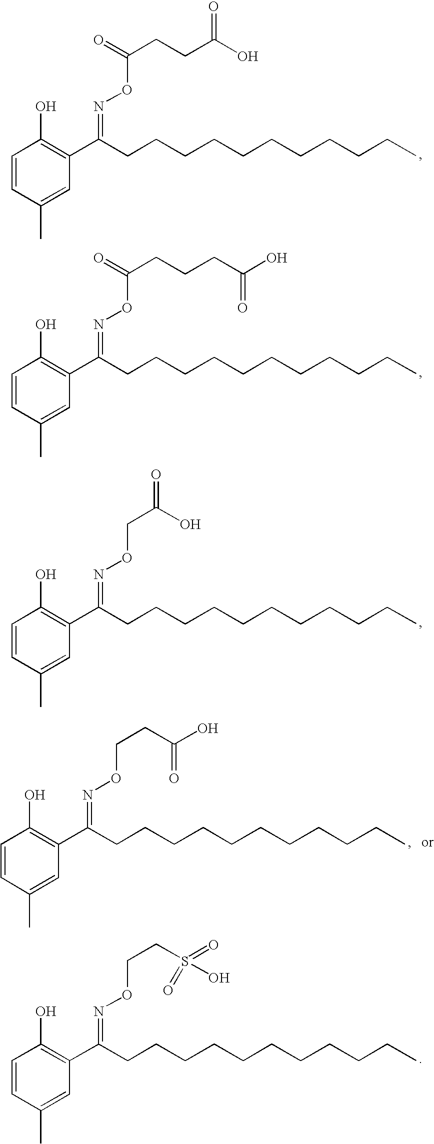

3. A topical composition according to claim 2, wherein 2-hydroxy-5-methyllaurophenone oxime and/or at least one of the oxime derivatives is present.

4. A topical composition according to claim 1, which comprises the aryl oxime(s) in a total amount of 0.001 to 5% by weight.

5. A topical composition according to claim 1, which comprises bisabolol in an amount of 0.05 to 30% by weight.

6. A topical composition according to claim 1, which further comprises at least one antioxidant and/or UV filter.

7. A topical composition according to claim 1, which further comprises one or more substance(s) having an inflammation-inhibiting action.

8. A topical composition according to claim 1, which further comprises one or more compatible solutes.

9. A mixture which is suitable for the preparation of a topical composition comprising bisabolol and at least one aryl oxime of formula (I) 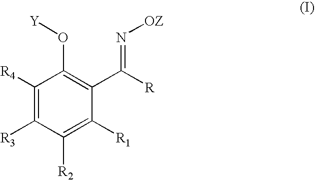
in which:
Y and Z, independently of one another, denote H, C₁₋₁₈-alkyl, C₂-₁₈-alkenyl, C₂₋₁₈-carboxyalkyl, C₃₋₁₈-carboxyalkenyl or C₂₋₁₈-alkanoyl;
R denotes C₁₋₁₈-alkyl, C₂₋₁₈-alkenyl, C₃₋₈-cycloalkyl, aryl, aralkyl, heteroaryl, heteroaralkyl or a fused system;
R₁, R₂, R₃ and R₄, independently of one another, denote H, C₁₋₁₂-alkyl, C₂₋₁₂-alkenyl, C₁₋₁₂-alkoxy, C₃₋₈-cycloalkoxy, aryl, aryloxy, aralkyl, heteroaryl, heteroaralkyl, carboxyl, hydroxyl, chlorine, dialkylamine or sulfonyl.

10. A mixture according to claim 9, which comprises 1 to 20% by weight aryl oxime(s) and 80 to 99% by weight bisabolol.

11. A mixture according to claim 10, which comprises 5 to 15% by weight aryl oxime(s) and 85 to 95% by weight bisabolol.

12. A mixture according to claim 9, which further comprises 5-30% by weight an additional oil, and comprising 5 to 15% by weight aryl oxime(s) and 35 to 94% by weight bisabolol.

13. A process for preparing a topical composition according to claim 1 comprising bringing together a compound of formula I, bisabolol and a topically acceptable carrier, or bringing together a topically acceptable carrier and a mixture containing a compound of formula I and bisabolol.

14. A method for the prophylaxis and/or treatment of a skin disease and/or an inflammation reaction of the skin comprising administering the topical composition of claim 1 to the skin.

15. A method for cosmetic care of skin comprising administering the topical composition of claim 1 to the skin.

16. A process according to claim 13, comprising bringing together a topically acceptable carrier and a mixture containing a compound of formula I and bisabolol.

17. A method according to claim 14, wherein inflammation is treated.
